(19) United States
(12) Patent Application Publication
(10) Pub. No.: US 2002/0095128 A1
(43) Pub. Date: Jul. 18, 2002

(54) Withdrawal String for a tampon

(76) Inventor: Michael G. Petit, (US)

Correspondence Address: Michael G. Petit, P. O. Box 91929, Santa Barbara, CA, 93190-1929, US

(21) Appl. No.: 09/875,516

(22) Filed: Jun. 5, 2001

Related U.S. Application Data
(63) Continuation-in-part of application No. 09/524,932, filed on Mar. 14, 2000, now abandoned.
Application No. 09/524,932 is a continuation-in-part of application No. 09/312,915, filed on May 17, 1999, now abandoned.

(51) Int. Cl. A61F 13/15

(52) U.S. Cl. 604/385.18; 604/904

(57) ABSTRACT

A withdrawal string for use with an absorbent pledget. The withdrawal string is affixed to an absorbent pledget which is insertable into a body cavity. The withdrawal string is a multifilamentous twine having a fixed end attached to the pledget and a free end. The multiple filaments comprising the withdrawal string are preferably hydrophobic and intertwined. At least one barrier portion is disposed between the free end and the fixed end of the withdrawal string. The purpose of the barrier portion is impede diffusion of autogenous and exogenous material, such as blood cells, bacteria and viruses, between the free end and the fixed end of the string when the string is wetted thereby reducing or eliminating material transport along the string. The barrier portion may be formed by heating, crimping, solvent fusion or, most preferably, by permeating one or more segments of the withdrawal string between the free end and the pledget with an uncured elastomer and curing the elastomer to provide a viscous diffusion barrier. When such a withdrawal string is wetted, such as, for example, when the string is immersed in a fluid medium such as a spa or swimming pool, the barrier portion renders the string effective for impeding the transport of blood to undergarments or exogenous fluids bearing particulates and the like into the pledget and/or vagina.

### BACKGROUND OF THE INVENTION

1. Field of the Invention

The invention relates generally to absorbent articles useful for collecting body fluids and more particularly to a withdrawal string for tampons.

2. Prior Art

It is suggested in the art that when a withdrawal string comprising twine is attached to an absorbent pledget as, for example, embodied by a tampon, the conduction of blood along the withdrawal string occurs when the pledget becomes saturated with menstrual blood. Conversely, if the pledget is not saturated, external (exogenous) fluids such as bath water may be wicked into the vagina via the withdrawal string. Comin-DuMong, in U.S. Pat. No. 5,458,589, discloses the use of a monofilamentous withdrawal string to reduce “wicking” of catamenial fluid. The catamenial tampon industry currently employs multifilamentous twine for tampon withdrawal strings and re-tooling would be required to manufacture tampons having a monofilamentous withdrawal string. In addition, the introduction of new, non-FDA approved materials for making a withdrawal string may require the performance of expensive safety and efficacy studies prior to receiving regulatory approval for the sale of tampons comprising such a string.

Jones, Sr., in U.S. Pat. No. 3,693,622, addresses the problem of preventing menstrual flow from the pledget to the vaginal opening. Jones, Sr. discloses that coating the trailing end of a tampon pledget (i.e., the end of the pledget in opposition to the insertion end) with a menstrual flow repellant such as dimethyl polysiloxane oil provides a means for preventing leakage of menstrual flow from the pledget. Such a solution does not, however, prevent or otherwise impede the wicking of an exogenous fluid into the vagina by the withdrawal string.

J. L. Jones, in U.S. Pat. No. 3,520,302, suggests using a bifilamentous cellulose rayon yarn for a withdrawal string. In order to attach the withdrawal string to an absorbant pledget, Jones suggests coating the string with a fusible material and, after bringing the coated string in contact with the pledget and compressing the assembly thus formed, heating the assembly above the fusing temperature of the coating. Thus, while the Jones withdrawal string comprising the fusible material is readily attached to a pledget, the string exhibits no other advantage related to wicking.

Gellert, in U.S. Pat. No. 4,475,911, and Rabell, in U.S. Pat. No. 2,146,985, disclose a withdrawal string that is either “moisture resistant” twine or a twine comprising a “waterproof coating” along the entire length thereof. Both withdrawal strings have a substantially homeogeneous composition when viewed longitudinally along the entire length thereof. A problem with applying a waterproof coating to the string is that it provides a different and unacceptable “feel” to the user and has not enjoyed commercial success in the market. The perception of a familiar “feel” by the user appears to be an important factor in consumer acceptance of a tampon.

Zadini et al., in U.S. Pat. No. 5,609,586, disclose a tampon wherein an expandable member is affixed to the pledget and the fixed end of the withdrawal string is affixed to the expandable member. The stated purpose of the expandable member is to provide an intravaginal seal to retain menstrual fluid within the vagina. The inflatable portion is not spaced from the pledget. The fixed end of the withdrawal string is affixed to the trailing end of the inflatable member (i.e., the end of the inflatable member closest to the vaginal opening) rather than to the pledget because in order to remove the device from the vagina, the inflatable member must first be elongated. This is done by applying traction is to the withdrawal string. Accordingly, the string must be attached to the trailing end of the expandable member rather than the leading end of the pledget. While the tampon thus formed may reduce leakage of menstrual fluid from the vagina, it does not reduce, impede or prevent the conduction of exogenous fluids into the vagina via the withdrawal string.

It would be particularly desirable to provide a withdrawal string for attachment to an absorbent pledget that impedes material transport along the string into the vagina under conditions wherein an extracorporeal “free” end of the string (i.e., the end of the withdrawal string opposite the pledget) is immersed in an exogenous fluid such as pool, spa or bath water. It is further desirable to provide a withdrawal string which may be made using a conventional twine and FDA-approved material and is perceived by the user as having a familiar feel. Preferably the tampon assembly incorporating such a withdrawal string can be made in accordance with prior art manufacturing methods.

### SUMMARY OF THE INVENTION

It is a first object of the invention to provide a withdrawal string having a free end and a fixed end adapted for attachment to an absorbent pledget wherein the withdrawal string substantially impedes transport of microorganisms suspended in an aqueous fluid in contact with the free end of the string to the fixed end.

It is another object of the invention to provide a catamenial tampon having a withdrawal string which when wetted with an aqueous fluid, impedes diffusion of blood components between the pledget and the free end of the string.

It is another object of the invention to provide a catamenial tampon having a withdrawal string which, when wetted with an exogenous aqueous fluid, impedes transport of blood components between the pledget and the free end of the string by capillary action.

It is another object of the invention to provide a catamenial tampon having a withdrawal string which, when wetted with an autogenous aqueous fluid, resists transport of blood components between the pledget and the free end of the string.

It is yet a further object of the invention to provide a withdrawal string comprising a twine and having a fixed end attached to an absorbent pledget, and a free end, the withdrawal string, when wetted, being resistant to diffusion of components suspended or dissolved in an exogenous aqueous fluid along the length of the string when the free end of the string is immersed in the aqueous fluid.

Prior art efforts to prevent fluid transport along the withdrawal string of a tampon have focused on transport by capillary action. Surprisingly, experiments conducted by the present inventor suggest that there is little or no material transport along a tampon withdrawal string when the string is dry. This is true even when the pledget to which the string is affixed is saturated with blood. However, when a tampon withdrawal string is wetted by an aqueous fluid, such as by immersion in water, there is substantial material transport along the length of the string which transport occurs between the free end of the string, which, in use, extends outwardly from the vagina, and the intravaginally disposed pledget. It is an object of the invention to provide a withdrawal string for a tampon which retards the transport of fluids and fluid-borne particulates along the length of the string even when the string is wet. The period of retardation is preferably sufficient to enable a user to enjoy immersion in a water bath for a period of at least an hour without substantial transport of suspended particulates, including cells and microorganisms, into or out of the vagina by a string-mediated transport mechanism.

The features of the invention believed to be novel are set forth with particularity in the appended claims. However the invention itself, both as to organization and method of operation, together with further objects and advantages thereof may be best be understood by reference to the following description taken in conjunction with the accompanying drawings in which:

### BRIEF DESCRIPTION OF THE DRAWINGS

FIG. 1 is an elevational view of a tampon in accordance with the prior art.

FIG. 2 is a cross-sectional view of the withdrawal string comprising twine taken along section line 2-2 of FIG. 1.

FIG. 3 is an elevational view of a tampon in accordance with the present invention.

FIG. 4 is cross-sectional view of a barrier portion of the withdrawal string of the tampon of FIG. 3 taken along section line 4-4 wherein FIGS. 4a-4c show a barrier portion formed by crimping with or without melting and FIG. 4d shows a barrier portion formed by injection of an elastomer into the interfiber spaces or “grooves” in a twine.

FIG. 5 illustrates the transport of a dye solution from a reservoir of dye solution in contact with the free end of a prior art tampon withdrawal string, which string has been wetted with water by immersion therein, to a wet pledget affixed to the opposing end of the withdrawal string.

FIG. 6 is similar to FIG. 5, illustrating the transport of a dye solution from a pledget saturated with the dye solution to the free end of a wetted withdrawal string in accordance with the prior art.

FIG. 7 is as FIG. 5 wherein the withdrawal string includes a diffusion barrier portion disposed between the free end and the fixed end of the withdrawal string which resists transport of the dye solution thereacross.

FIG. 8 is the experimental set up shown in FIG. 6 wherein the withdrawal string has been modified in accordance with the present invention to include a barrier portion disposed between the free end and the fixed end of the withdrawal string which resists transport of the dye solution thereacross.

FIG. 9 is a cross-sectional view of a withdrawal string, taken along section line 2-2 of FIG. 1, wherein the withdrawal string is comprised of two intertwined fibers, or intertwined portions of a single fiber, each of which fiber portions are comprised of a plurality of intertwined monofilamentous fibers.

FIG. 10 is as shown in FIG. 9 wherein the spaces between adjacent fibers, both multifilamentous and monofilamentous, are filled with an elastomer to form a barrier portion in the withdrawal string which impedes fluid transport between the free end and fixed end of the string.

### DESCRIPTION OF THE PREFERRED EMBODIMENTS

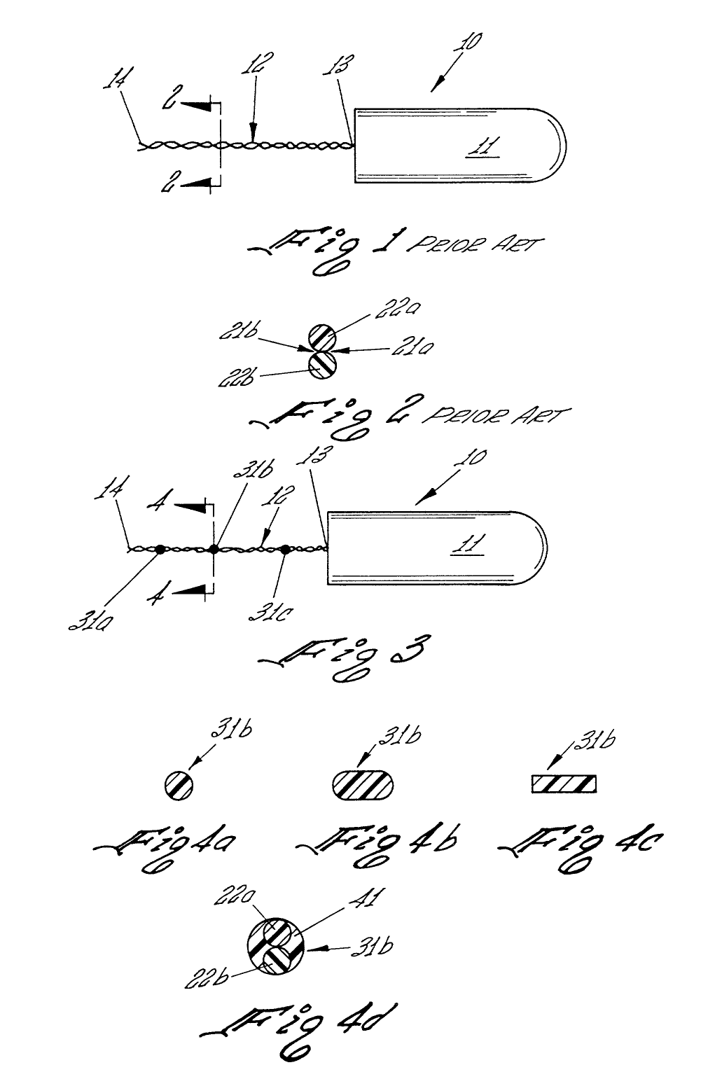

Turning now to FIG. 1, a tampon 10, in accordance with the prior art, is shown in horizontal elevational view. An absorbent pledget 11 is attached to a withdrawal string 12 having a fixed end 13 and a free end 14. The absorbent pledget 11 comprises a hydrophilic body having an indefinite composition that includes at least one material which absorbs aqueous fluids such as cotton, silk, or rayon fibers, shaped into a desired form. Between the free end 14 and the fixed end 13, the withdrawal string comprises a twine. The term “twine”, as used herein, means an elongate member comprising 2 or more strands or fibers which are twisted together. The fibers comprising the twine may also be twines; consisting of a plurality of intertwined monofilamentous fibers. The withdrawal string of the present invention comprises at least one twine. While the fibers comprising the twine are preferably hydrophobic, they may also be hydrophilic without departing from the objects of the invention..

A cross-sectional view of the withdrawal string 12 is shown in FIG. 2. Two more or less helical fluid conducting channels or grooves 21a and 21b are present in the interfiber spaces between the two fibers 22a and 22b comprising the withdrawal string 12. It is understood that in the event that the fibers 22a and 22b are themselves comprised of a plurality of intertwined fibers (such as intertwined monofilamentous subfibers), there will also be a plurality of elongate, helical spaces or “subgrooves” between the intertwined subfibers comprising the fibers 22a and 22b. Such a plurality of subgrooves, some of which are illustrated in cross-sectional view at 90a-f in FIG. 9, also provide conduits for fluid transport. Accordingly, the present invention additionally contemplates the closure of all such subgrooves to fluid transport. In the following discussion, a “barrier portion” of a withdrawal string is taken to mean a segment of a tampon withdrawal string, the segment being disposed between the free end and the fixed end thereof, which has been modified in such a way as to impede fluid transport thereacross.

Returning now to FIG. 2, when a tampon 10 is inserted within a vagina (not shown) and the withdrawal string 12 projecting therefrom is immersed in an aqueous medium, such as bath water or vaginal secretions, the helical grooves 21a and 21b fill with fluid and the withdrawal string becomes wetted by immersion in the fluid. The plurality of grooves 21a and 21b, are substantially coextensive with the withdrawal string and become a plurality of fluid-filled conduits enabling the transport of the wetting fluid, or a material suspended or dissolved in the fluid, or any other fluid in contact with a portion of the withdrawal string 12, along the length of the withdrawal string. Such material transport along the length of the withdrawal string may allow blood, or components thereof which are present in a saturated pledget 11, to be transported to the free end and, in turn, to objects such as bath water or dry undergarments (not shown) that are in contact with the free end 14 of the withdrawal string via diffusion and/or capillary action.

FIG. 3 is an elevational view of a tampon 10 wherein the withdrawal string 12 has been modified by removing a portion of the helical grooves 21a and 21b, preferably by filling them with a viscous elastomer or a fusible material, in one or more segments of the string (in this example, three barrier portions are present) to form barrier portions 31a, 31b, and 31c. The barrier portion(s) are disposed between the free end 14 and the fixed end 13 of the withdrawal string 12. A cross-sectional view of an exemplary barrier portion 31b of the withdrawal string 12 viewed along section line 4-4 is shown in FIGS. 4a-4d. In FIGS. 4a, 4b and 4c, the barrier portion 31b the individual fibers 22a and 22b comprising the twine have been fused by chemical and/or physical means such as by melting and/or crimping to fill or otherwise remove the grooves 21a and 21b between the fibers thereby interrupting the grooves and forming a barrier for material transport between the free end 14 of the withdrawal string and the fixed end 13 as shown in FIGS. 4a, 4b and 4c. The term “barrier portion”, as used herein, means a portion of the withdrawal string that is more resistant to fluid conduction along the length of the string than portions of the string adjacent thereto. While compression of the twine comprising the withdrawal string, with or without the application of heat or solvent, is one method for making a barrier portion, other methods may be employed. For example, in a preferred embodiment, a hydrophobic elastomer 41, such as uncured or partially cured silicone elastomer, may be injected into the grooves to fill and eliminate at least a portion of the grooves as shown in FIG. 4d. In a further embodiment, a solvent or catalytic agent capable of melting or cross-linking a component of the polymer comprising the twine may be used to soften and reshape a portion of the twine to eliminate the grooves and provide a barrier portion. Silicone rubber is a particularly preferred elastomer for use in forming a barrier portion due to its hydrophobicity, hypoallergenicity, low toxicity, and extensive history of safety in such products as vaginal diaphragms, cervical caps, condoms and medical implants.

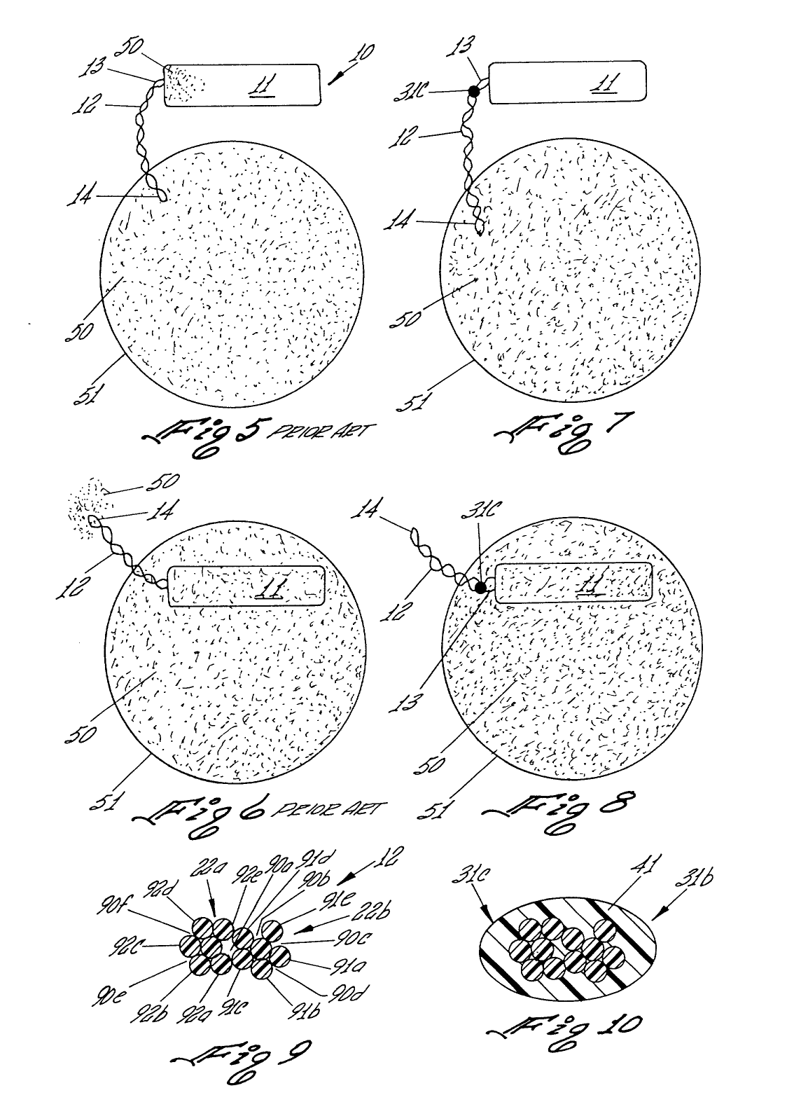

The effectiveness of silicone elastomer for forming a barrier portion on a segment of a withdrawal string is illustrated by reference to FIGS. 5-8. In this experiment, a dye solution 50 comprising blue food coloring in water was prepared and placed in a shallow tray 51 comprising a dye reservoir. Surprisingly, when the free end 14 of a dry withdrawal string 12 comprising a prior art tampon 10 was immersed in the dye solution 50 in accordance with the experimental set-up shown in FIG. 5, no dye transport was observed along the length of string, even after 24 hours of immersion in the dye solution. However, when the experiment was repeated by first immersing the pledget 11 and withdrawal string 12 in clear water before placing the free end 14 in contact with the dye solution 50 contained in the dye reservoir, the dye 50 was quickly transported along the string 12 and was taken up by the pledget 11 as shown in FIG. 5. This effect was observed whenever the withdrawal string and the pledget of the prior art tampon were wetted by pre-soaking the tampon in water. FIG. 5 illustrates the transport of the dye solution from the free end 14 of a prior art tampon withdrawal string, which has been wetted as described above by immersion in clear water, to a wet pledget 11 affixed to the opposing end of the withdrawal string 12. The experiment illustrated in FIG. 6 is similar to FIG. 5, but reversed, showing the transport of the dye solution 50 from a pledget 11 saturated with the dye solution 50 to the free end 14 of a wetted withdrawal string 12 comprising a prior art tampon 10.

The experiment described above, and illustrated in FIGS. 5 and 6, was repeated after first permeating and circumscribing a short (2 mm long) segment of the withdrawal string 12 comprising the prior art tampon (i.e. the same brand of prior art tampon used in the experiments shown in FIGS. 5 and 6), with uncured RTV silicone elastomer (Dow Chemical Co.) and allowing the elastomer to cure at room temperature. The silicone-treated segment 31c, after the silicone was cured, formed a 2 mm long barrier portion disposed about two centimeters from the withdrawal string's fixed end. FIG. 7 shows the result of duplicating the experiment shown in FIG. 5 when the withdrawal string includes a barrier portion 31c disposed between the free end and the fixed end of the withdrawal string. The inclusion of a barrier portion 31c in at least one segment of the length of the withdrawal string 12 prevents the transport of the dye solution thereacross.

FIG. 8 duplicates the experimental set-up shown in FIG. 6, but wherein the withdrawal string 12 has been modified by the inclusion of a silicone barrier portion 31c in the withdrawal string 12 as describe above in FIG. 7, and in accordance with the preferred embodiment of the present invention. The incorporation of a silicone barrier portion 31c within the withdrawal string 12 between the free end 14 and the fixed end 13 thereof and disposed about one and one half centimeters from the fixed end of the string, impedes material transport of the aqueous dye solution thereacross, even when the withdrawal string has been wetted along its entire length by submersion in an aqueous fluid.

In the event that the withdrawal string comprises a plurality of interwoven fibers wherein the fibers comprise interwoven monofilaments, the spaces between the monofilaments comprising the fiber provide a plurality of fluid conducting conduits as shown in FIG. 9. In such an event the cross-sectional view along section line 2-2 of FIG. 1 will appear as shown in FIG. 9. The monofilamentous fibers 91a-e and 92a-e comprising fibers 22a and 22b have a plurality of helical conduits therebetween, some of which are shown at 90a-f. In the preferred embodiment of the invention, a segment of the withdrawal string having the cross-section shown in FIG. 9 is permeated with uncured or partially cured silicone elastomer over a small portion of the length of the withdrawal string, preferably 1-10 mm, until the silicone 41 fills the spaces 90a-f between the monofilaments and the grooves around the perimeter of the segment of withdrawal string as shown in FIG. 10. After curing, the silicone elastomer 41 fills, or substantially fills the interstices and provides a barrier portion that substantially impedes fluid conduction within the spaces.

In summary, the preferred embodiment of a catamenial tampon, in accordance with the present invention, comprises an absorbent pledget, adapted for insertion into a vagina, affixed to a fixed end of a withdrawal string comprising twine. The withdrawal string has a free end in opposition to to the fixed end and has a length therebetween. A segment of the length of the withdrawal string is a barrier portion operable for impeding the transport of an aqueous fluid between the free end of the withdrawal string and the fixed end when the length of the withdrawal string adjacent to the barrier portion is wetted with an aqueous fluid. The segment of the length of the withdrawal string comprising the barrier portion includes a plurality of adjacent, substantially parallel, coextensive fibers having spaces therebetween which spaces are filled with a cured or partially cured elastomer such as silicone. The barrier portion preferably further includes an outer layer of cured silicone elastomer or hydrophobic thermoplastic material overlying the segment of length comprising the barrier portion of the withdrawal string. The length of the segment comprising the barrier portion, and hence the length of the barrier portion, is less than the length of the withdrawal string and preferably in the range of between 1-10 mm in length. Alternatively, the spaces between adjacent fibers comprising the barrier portion are removed by compression of the segment or fusion of the fibers by chemical or physical means.

Thus, it has been shown that the incorporation of a fluid barrier within a segment of the length of a tampon withdrawal string comprised of a twine impedes material transport between the free end and the fixed end of the withdrawal string, even when the entire length of the withdrawal string is wetted with an aqueous fluid. Such a wetted condition may result, for example, from immersion of the withdrawal string in bath water or by an errant stream of urine impinging thereon. In addition, the present invention provides means for modifying a prior art withdrawal string that has an established historical safety record, to improve the fluid conduction characteristics thereof without the need for incorporating untested materials therein or substantial retooling a manufacturing facility.

While particular embodiments of the present invention have been illustrated and described, it would be obvious to those skilled in the art that various other changes and modifications can be made without departing from the spirit and scope of the invention. For example, a barrier portion of a tampon withdrawal string may comprise a single short segment or multiple short segments of the length of the withdrawal string. The elastomer may be silicone, polyurethane or a similar biocompatible elastomer material. It is therefore intended to cover in the appended claims all such changes and modifications that are within the scope of this invention.

What I claim is:

1. A withdrawal string for a tampon, the withdrawal string comprising a plurality of elongate, substantially coextensive, intertwined fibers and having a fixed end attached to an absorbent pledget, a free end in opposition to said fixed end, and a length therebetween wherein a portion of said length is a barrier portion operable for impeding the transport of an exogenous aqueous fluid along said length between said free end of said withdrawal string and said fixed end of said withdrawal string when said free end is in fluid contact with the exogenous fluid, said portion of said length comprising said barrier portion being spaced from said free end of said withdrawal string.

2. The withdrawal string of claim 1 wherein said barrier portion is spaced from said fixed end of said withdrawal string.

3. The withdrawal string of claim 1 wherein said barrier portion comprises a hydrophobic elastomer layer coextensive with said barrier portion.

4. The withdrawal string of claim 1 wherein said fibers comprise a hydrophobic material.

5. The withdrawal string of claim 2 wherein said fibers comprise a hydrophobic material.

6. A catamenial tampon comprising an absorbent pledget adapted for insertion into a vagina and a withdrawal string comprising twine, the withdrawal string having a fixed end affixed to said pledget and a free end in opposition to said fixed end and a length therebetween, wherein a portion of said length of said withdrawal string is a barrier portion, said portion of said length comprising said barrier portion being spaced from said pledget and said free end, said barrier portion being operable for impeding the transport of an aqueous fluid between said free end of said withdrawal string and said pledget when said free end of said withdrawal string is wetted with an aqueous fluid.

7. A catamenial tampon in accordance with claim 6 wherein a segment of said length of said withdrawal string comprises a plurality of coextensive fibers having spaces therebetween and wherein said barrier portion comprises an outer layer of cured elastomer overlying said segment of said length of said withdrawal string and filling said spaces between fibers underlying said outer layer.

8. The catamenial tampon of claim 6 wherein said segment has a segment length which is less than said length of said withdrawal string.

9. A catamenial tampon in accordance with claim 6 wherein a segment of said length of said withdrawal string comprises a plurality of coextensive fibers having spaces therebetween and wherein said barrier portion is formed by compressing said segment to remove said spaces between said coextensive fibers.
